(19) United States
(12) Patent Application Publication
(10) Pub. No.: US 2025/0161348 A1
(43) Pub. Date: May 22, 2025

(54) PROCESS FOR PREPARATION OF COMPOSITIONS OF OLIGOMERS OF LACTIC ACID

(71) Applicant: Combe Incorporated, White Plains, NY (US)

(72) Inventors: Gabriella YOUSEF, Sundbyberg (SE); Jeanette FOLKING, Helsingborg (SE)

(21) Appl. No.: 18/840,535

(22) PCT Filed: Feb. 28, 2023
(86) PCT No.: PCT/EP2023/054960

§ 371 Date: Aug. 22, 2024

(30) Foreign Application Priority Data
Mar. 1, 2022 (EP) 22159316.3

(51) Int. Cl. A61K 31/765; A61K 9/20

(52) U.S. Cl. CPC A61K 31/765; A61K 9/2018; A61K 9/2054; A61K 9/2095

(57) ABSTRACT

A process for the preparation of a composition comprising oligomers of lactic acid as active substance, which includes:
i) premixing pharmaceutically acceptable excipients comprising one or more selected from binders and fillers,
ii) a first wet granulation step wherein an aqueous solution comprising oligomers of lactic acid is added to the premix obtained in step i),
iii) milling the wet first granulate obtained from step ii),
iv) drying the milled wet first granulate obtained from step iii) and dry milling the dry granulate,
v) a second wet granulation step wherein an aqueous solution comprising oligomers of lactic acid is added to the dried first granulate from step iv),
vi) milling the wet second granulate obtained from step v),
vii) drying the wet second granulate obtained from step vi), and
viii) milling the dried second granulate obtained from step vii) to obtain a particulate material comprising oligomers of lactic acid.

### FIELD OF THE INVENTION

The present invention relates to a process for the preparation of compositions of oligomeric lactic acid. The compositions are in solid form and include both particulate material as well as dosage form like tablets, capsules etc. The particulate material may be further processed eg into such dosage forms.

The dosage forms are suitable for use in the treatment or prophylaxis of gynecological infections. The infection may be a bacterial infection, a viral infection or a fungal infection.

### BACKGROUND

The present invention is a development of the applicant's previous applications WO 2008/119518 and WO 2014/012805. Both applications relate to the treatment or prophylaxis of gynecological infections using oligomers of lactic acid.

Gynaecological or reproductive tract infections generally refer to three different types of infection which affect the reproductive tract. Endogenous infections include bacterial vaginosis and candidosis, which result from an overgrowth of organisms which are normally present in the vagina. The endogenous infections represent the most common form of lower gynaecological tract infections (LGTIs) worldwide, and they can be easily treated. However, they commonly reappear, which is a major medical problem. Latrogenic infections represent a second group which occur when the infectious agent (a bacterium or other micro-organism) is introduced into the reproductive tract through various routes such as menstrual regulation, induced abortion, IUD insertion or during parturition. Finally, sexually transmitted infections (STIs) are caused by microorganisms such as viruses, bacteria, or parasitic microorganisms that are transmitted through sexual activity with an infected partner. Among the STIs there are several serious diseases such as HIV, Chlamydia trachomatis, condyloma accuminata, syphilis and Neisseria gonorrhoea. STIs can affect both men and women, but a transmission from mothers to children during pregnancy and childbirth may also occur.

Bacterial vaginosis (BV) is the most frequent endogenous infection and also the most common medical condition of the female genital tract. BV is linked to increased complications in pregnancy and may be involved in the pathogenesis of pelvic inflammatory disease and women's risk of acquiring HIV. Still many questions remain about its aetiology, which complicates the management of recurrent infections.

BV is an overgrowth of anaerobic bacteria and a lack of normal Lactobacilli flora, which results in an imbalance of normal vaginal flora. During pregnancy BV is associated with poor perinatal outcome and a cause of preterm birth. Identification and treatment of BV may reduce the risk of such consequences. A range of therapeutic options has been tested in order to manage or prevent recurrences of BV.

It is not yet known whether frequent episodes of BV are the result of re-infection or relapse. The association of BV with sexual behaviour suggests that BV is sexually transmitted and that additional episodes may be due to re-infection. However, evidence do not support the theory of sexual transmission and re-infection and several studies evaluating risk factors for repeated episodes of BV suggest it is due to relapse. Women developing early recurrence tend to complain of abnormal discharge at the end of therapy. Moreover, asymptomatic women who consider themselves cured after treatment, continued to have abnormal vaginal flora. Furthermore, the more severe the abnormality the earlier is usually the recurrence.

The value of bacteriotherapy, using harmless bacteria to displace pathogenic organisms remain unresolved.

Psychosexual symptoms with lack of libido and anxiety about infection may be reported by some women as a consequence of recurrent episodes of bacterial vaginosis and associated malodour. However, concurrent treatment of the male partner does not reduce the rate of BV relapse. However, condom use with male sexual partners may help to reduce the risk of relapse of bacterial vaginosis. Hormonal contraception use does not increase the incidence of bacterial vaginosis, 30 while women with an intrauterine contraceptive device or system in situ may have an increased risk of BV.

### Vaginal Discharge

Vaginal discharge is a common presenting symptom, which may be physiological or pathological. While BV remains one of the most common diagnoses in women attending genitourinary medicine clinics, vulvovaginal candidiasis is another common infective cause of vaginal discharge that affects about 75% of women at some time during their reproductive life. Approximately 50% of cases of bacterial vaginosis are asymptomatic and the true prevalence of this condition in the community is about 10-30%. Lactobacilli colonising the vaginal epithelium may have a role in defence against infection. Normal vaginal flora (lactobacilli) maintains the vaginal pH between 3.8 and 4.4. The quality and quantity of vaginal discharge may be altered in the same woman over time. There is a wide variation in vaginal discharge and each woman has her own sense of normality and what is acceptable or excessive.

The main problem of the pathogenic vaginal discharge is the malodour. This odour has the characteristics of a foul fishy smell which is characteristic for bacterial vaginosis and caused by amines, mainly trimethylamine. Other clinical manifestations may be excessive discharge and a sense of unfreshness.

As mentioned above, the present invention is a further development of the technologies described in the applications referred to above. The inventors have developed an improved method for the preparation of compositions comprising oligomers of lactic acid. The overall problem observed with oligomers of lactic acid is the sticking effect, which makes it difficult to control the manufacturing processes as the oligomers of lactic acid optionally admixed with pharmaceutically acceptable excipients tend to adhere to the surface of the manufacturing equipment. This makes it difficult to achieve a reproducible amount of oligomeric lactic acid in the final product. However, the final product should—when vaginally administered—have a certain bioadhesiveness, Thus, it is important to develop a process where stickiness to the manufacturing equipment is avoided without deteriorating the possibility of obtaining a composition that is bioadhesive upon application. Moreover, the composition should have a suitable stability and shelf-life.

### SUMMARY

Oligomeric lactic acid, which is the active substance, is very sensitive to water and will be degraded into smaller fragments, to lactate or to lactic acid. Moreover, oligomeric lactic acid—when contacted with water—has a tendency to stick to surfaces. A composition of the invention is intended to be administered vaginally and therefore, it should be able to adhere to the vaginal mucosa so that the composition remains at the application site for a suitable period of time. Thus, it is advantageous that the oligomeric lactic acid when contacted with a body fluid becomes sticky, but on the other hand it should be possible to process the oligomeric lactic acid into a composition suitable for vaginal administration without experience stickiness to the manufacturing apparatus.

The present inventors have developed a process for the preparation of an oligomeric lactic acid composition, where the water content in controlled in the steps involved in the process. To avoid over-wetting of the powder, the process involves two wet granulation steps separated by a drying and milling step. In this manner the water content of the material in each step can be controlled.

As mentioned above, the water content is important to control during the manufacturing process to avoid sticking to surfaces such as surfaces of the apparatus employed. It should, however, be noted that when the tablet has been applied to a mucosal surface such as the vaginal mucosa, the body fluid present on the mucosa will impart adhesiveness/stickiness to the tablet so that it can adhere to the mucosa and stay on the mucosa for a desired period of time.

Accordingly, the present invention provides a process for the preparation of a composition comprising oligomers of lactic acid as active substance, the process comprising the steps of:
i) premixing pharmaceutically acceptable excipients comprising one or more selected from binders and fillers,
ii) a first wet granulation step wherein an aqueous solution comprising oligomers of lactic acid is added to the premix obtained in step i),
iii) milling the wet first granulate obtained from step ii),
iv) drying the milled wet first granulate obtained from step iii) and dry milling the dry first granulate,
v) a second wet granulation step wherein an aqueous solution comprising oligomers of lactic acid is added to the dried first granulate from step iv),
vi) milling the wet second granulate obtained from step v),
vii) drying the wet second granulate obtained from step vi), and
viii) milling the dried second granulate obtained from step vii) to obtain a particulate material comprising oligomers of lactic acid.

The particulate material obtained may be used as such, it may be admixed with further acceptable excipient and it may be processed into a dosage form.

A suitable dosage form for vaginal administration includes tablets.

Tablet compositions prepared according to the invention has been tested in stability tests both a room temperature and in accelerated studies. 6 months stability testing at 25° C./60% RH, 40° C./75% RH and 50° C./75% RH showed excellent stability for the compositions tested with respect to visual inspection, mean tablet weight, content of lactic acid, degree of polymerization, pH, water content and dissolution profiled measures as % lactic acid dissolved/released at 6 hours, 24 hours, 48 hours and 72 hours after start of the dissolution testing.

### DETAILED DESCRIPTION

The present invention provides a process for the preparation of a composition comprising oligomers of lactic acid as active substance, the process comprising the steps of:
i) premixing pharmaceutically acceptable excipients comprising one or more selected from binders and fillers,
ii) a first wet granulation step wherein an aqueous solution comprising oligomers of lactic acid is added to the premix obtained in step i),
iii) milling the wet first granulate obtained from step ii),
iv) drying the milled wet first granulate obtained from step iii) and dry milling the dry first granulate,
v) a second wet granulation step wherein an aqueous solution comprising oligomers of lactic acid is added to the dried first granulate from step iv),
vi) milling the wet second granulate obtained from step v)
vii) drying the wet second granulate obtained from step vi), and
viii) milling the dried second granulate obtained from step vii) to obtain a particulate material comprising oligomers of lactic acid.

The process controls the water content in the individual step in order to reduce any negative impact from the water on i) the stability of oligomeric lactic acid in the individual steps, ii) the stability and shelf-life of oligomeric lactic acid in the final product, and i) the adherence of the intermediate mixtures and resulting product to the manufacturing equipment. Moreover, a two-step granulation procedure, where milling and drying steps are steps between the two granulation steps, ensure a suitable particle size distribution in the particulate material obtained. A suitable particle size distribution is a particle size distribution that is not too narrow. D50 (μm) should be in a range of from about 100 to about 350 μm such as from about 150 to about 325 μm or from about 200 to about 300 μm, but some of the particle should have a size of 200 μm or more. A broad particle size distribution has been observed to be beneficial for the result of the compression into tablets. The particle size distribution is typically measured by means of laser diffraction using Malvern, Mastersizer 3000. in order to ensure the possibility of obtaining a high-weight tablet when the particulate material is further compressed into tablet, i.e. a tablet wherein the weight is relatively high compared with the size of the tablet. Thus, the particulate material obtained should have a high powder density

Step i) of the process of the invention is typically carried out in a suitable apparatus such as a shear mixer or a fluid bed. Step i) involves premixing pharmaceutically acceptable excipients comprising one or more selected from binders and fillers. The mixing is typically carried out at room temperature. Before mixing the pharmaceutically acceptable excipients used may be dried at elevated temperature to reduce the water content.

In the present context, the term “pharmaceutically acceptable excipient” is intended to denote any material, which is inert in the sense that it substantially does not have any therapeutic and/or prophylactic effect per se. A pharmaceutically acceptable excipient may be added to the active drug substance with the purpose of making it possible to obtain a pharmaceutical composition.

Pharmaceutically acceptable excipients include those normally used in formulation of solid dosage forms such as e.g. fillers, binders, lubricants, modified-release agents, gelling or swelling agents, pH adjusting agents, stabilizing agents, coloring agents etc.

A binder is an important excipient in manufacturing of e.g. pellets, granules, spheres, minitablet, tablets and capsules. In some cases, crystalline forms of a drug substance can be directly compressed into a tablet, but the majority of drug substances must be converted into granules or pellets in order to obtain a sufficient cohesiveness so that it is possible to process the material e.g. to a solid dosage form. Binders hold the ingredients of a formulation together, for example in a tablet. Binders ensure that tablets, powders, granules and others can be formed with the required mechanical strength. Moreover, they give volume to low active dose tablets.

Mechanism of binding: When a suspension or a solution containing a binder is added to a mixture of powders, there is formation of liquid bridges and adsorption bonds between the particles and tensile of their bond increased (intergranular bonds). Surface tension forces and capillary pressure play crucial role in granule strength and formation. After the addition of binding agents adsorption bonds become active between particles surfaces in whole mixture which are
Binder-binder
Substrate-substrate
Binder-substrate

In some formulations binders are used in dry form and in others they are used as liquids by making a solution or suspension in suitable solvent like water, alcohol or mixture of these two. The proportion of binding agent varies from formulation to formulation. Tablets which are required to keep intact for a long time like lozenges and implants need high percentage of binders and which are to be disintegrate immediately need low quantity of binders for example effervescent tablets.

In a process of the present invention, a binder is not pre-dissolved or pre-suspended in a granulation liquid before addition to a powder blend. The binder is admixed with one or more pharmaceutically acceptable excipients and then followed by two granulation steps involving addition of a granulation liquid in the form of an aqueous solution comprising the active drug substance, i.e. oligomers of lactic acid, and a lactate. This premixing step is performed in order to control the water content in the product at the various steps involved in the process.

The primary criterion when choosing a binder is its compatibility with other tablet components.

Different types of binders are used in granulation: Natural binders: Starches including potato starch, maize starch and rice starch, modified starches, pregelatinized starch, acacia, gummi arabicum, tragacanth, alginic acid, alginate, cellulose, gelatin.

Synthetic or semi-synthetic binders: Methyl cellulose, ethyl cellulose, hydroxypropyl methylcellulose, microcrystalline cellulose, polyvinyl pyrollidine (PVP) polyvinyl alcohols, ploymethacrylates.

Sugars: Sucrose, liquid glucose, polyols such as sorbitol, mannitol, erythritol, tagatose, lactose, dextrins, maltodextrin.

Natural gums such as tragacanth and acacia are much effective when they are added in solution form. Synthetic polymers like PVP is used in alcohol gives granules which requires very less drying time and have good compressibility. It is used for granulation of moisture sensitive chewable and effervescent tablets. Starch is used as binding agent in wet granulation.

In the following table is given the generally applicable concentration of a binder in the final composition.

| | Recommended |
|---|---|
| Binder | percentage |
| Acacia gum with water or hydro alcohol | 2 to 5% |
| Gum Tragacanth with water | 1 to 3% |
| Gelatin with water | 1 to 4% |
| Sucrose with water | 2 to 20% |
| Starch in water | 1 to 4% |
| Sodium alginate with water | 3 to 5% |
| Methyl cellulose with water | 2 to 6% |
| Sodium caboxymethyl cellulose with water | 6% |
| Ethyl cellulose with alcohol | 0.5 to 2% |
| Hydroxylpropyl methylcellulose with water, | 2 to 5% |
| hydroalcohol, methylene chloride | |
| Polyvinyl pyrollidine with water, alcohol, | 0.5 to 5% |
| hydroalcohol | |
| Aluminium magnesium silicates with water | 2 to 10% |

According, the concentration of the binder in the final composition is generally in a range of from 0.5% to about 20% w/w. For cellulose derivatives, alginates, starches, polyvinyl pyrrolidone and polyvinyl alcohol the concentration in the final composition is generally in a range of from 0.5% to about 5% w/w.

However, in the event that a binder is used, which also may function e,g. as a filler or as a modifying release agent, the concentration of such a substance is higher as it not only function as a binder. Cellulose derivatives, alginates, starches, polyvinyl pyrrolidone (e.g. different grades of Kollidon®), povidone, and polyvinyl alcohol may also have impact on the release properties of the oligomeric lactic acid and can be used in concentrations that are 5-15 times higher than those indicated in the table above. Accordingly, if a binder is chosen to obtain binding properties and one or more other properties, the concentration of the binder in the final composition may be from about 10% to about 35% w/w such as from about 15% to about 30% w/w or from about 20% to about 30% w/w.

In a process according to the invention it is preferred to use a binder that also have modified release function. Thus, a binder for use according to the invention is typically selected from cellulose derivatives, alginates, starches, polyvinyl pyrrolidone and polyvinyl alcohol. The concentration of the binder in the final composition is from about 10% to about 35% w/w such as from about 15% to about 30% w/w or from about 20% to about 30% w/w.

A composition prepared by the process of the invention is intended to release lactic acid over a period of 8 hours or more such as 12 hours or more, 16 hours or more, 20 hours or more, 24 hours or more, 2 days or more, 3 days or more, 4 days or more when exposed to a dissolution and drug release testing of L-lactic acid oligomer in vaginal tablet. The in-vitro dissolution is performed in 900 mL 0.9% NaCl at 37° C. using paddles set at 100 rpm. At fixed time intervals, 0.5 M NaOH is added to raise the pH to 5.0-5.5. The dissolution is followed for from 2 to 96 h and is evaluated as total amount of base added divided by the theoretical amount needed to hydrolyze one vaginal tablet.

A binder may be selected that only is used for its binding function (although it may have other functions as well). In such a situation, a modified release agent is added to step i) of the process. Such modified release agents include cellulose derivatives including ethylcellulose, hydroxypropyl methylcellulose, sodium carboxymethylcellulose, hydroxypropyl methyl cellulose phthalate, cellulose acetate phthalate; acrylates, acrylic acid derivatives, gelatin, co-polymers based on polymethacrylic acid and methacrylates, ethyl acrylate and methyl acrylate, co-polymers of acrylic and methacrylic acid esters, polyvinyl acetate phthalate, fatty acids, fatty acid esters, fatty alcohols, cetyl alcohol, stearyl alcohol, and mixtures thereof.

The concentration of a modified release agent is in a range of from 5 to 40% w/w such as from 10% to 35% w/w, from 15% to 30%, from 20% to 30% w/w based on the total weight of the composition.

Normally, a filler is also included in the premix (step i). A filler is typically included to impart suitable technical properties to a powder such as improve flowability of the powder or particulate material. It may also be added if the amount of active drug substance is small so that it is difficult to manufacture a pharmaceutical composition due to a too low amount of material.

Fillers suitable for use according to the invention include: lactose (e.g. spray-dried lactose, alpha lactose, beta-lactose, lactose monohydrate, lactose monohydrate rennet free, Tabletose®, various grades of Pharmatose®, Microtose® or Fast-Floc®), cellulose, cellulose derivatives including hydroxypropyl cellulose, low-substituted hydroxypropyl cellulose, hydroxypropyl methylcellulose, methylcellulose, carboxyalkyl cellulose, microcrystalline cellulose (such as various grades of Avicel®: Avicel® PH101, Avicel® 102 or Avicel® 105; or Emcocel®, Vivacel®, Ming Tai®, Solka-Floc®); starches or modified starches (e.g. potato starch, maize starch, rice starch, pregelatinized starch), alginate (sodium alginate), calcium hydrogen phosphate, calcium phosphate, dibasic calcium phosphate, sodium phosphate, calcium sulfate, sodium carbonate, magnesium carbonate, magnesium chloride, polyethylene glycol, dextran soy polysaccharide, and the like, or mixtures thereof.

A filler is typically present in a composition of the invention in a concentration in a range of from about 20% to about 70% w/w such as from about 25% w/w to about 65% w/w, from about 30% to about 60% w/w, from about 35% to about 55% w/w, from about 40% to about 55% w/w.

Step ii) of the process of the invention is a first wet granulation step wherein an aqueous solution comprising oligomers of lactic acid is added to the premix obtained in step i),

Dividing the granulation step into two separate steps has been shown to be important in order to controlling the water content of the material. Thus, if the granulation is carried out in only one step there is a risk of over-wetting the material which result in degradation of the oligomeric lactic acid and sticking of the material to the manufacturing equipment resulting in variable, un-foreseen and non-reproducible content of oligomeric lactic acid in the final product.

Even if the granulation is performed in two separate steps, the ingredients in the granulation liquid used in the two steps preferably is the same.

The granulation liquid comprises the active substance, i.e. the oligomeric lactic acid, a pH regulating agent and water. Preferably, the granulation is an aqueous solution. The granulation liquid is prepared by dissolving the oligomeric acid in an aqueous solution of the pH regulating agent. The aqueous solution may be heated to a temperature of from about 30° C. to about 55° C. such as about 50° C. It seems as if the use of a granulation liquid at an elevated temperature (such as described above) makes it easier to add the granulation liquid to the powder bend as the viscosity decreases.

The pH regulating agent is added in order to obtain a pH value in a range of from about 2.5 to about 5.0 such as from about 2.7 to about 4.5 or from about 3.0 to about 4.0 or from about 3.1 to about 3.6, the target value being about 3.5. The pH of the final composition is measured by immersing the composition in water and measuring the pH. The pH should be to the acidic side. A low pH of the vagina is due to production of lactic acid by lactobacilli metabolism as well as conversion of glycogen to lactic acid by oestrogenised vaginal epithelial cells. In culture, lactobacilli acidify their growth medium to a pH of 3.2 to 4.8. At that pH range a steady state of equilibrium develops where the acidity becomes auto-inhibitory. Anaerobes grow poorly at pH 4.5 or less. In vitro studies show that the concentrations of BV associated bacteria increase with increasing vaginal pH. It has been found that lactic acid and low pH caused inhibitory effect on these bacteria better than hydrogen peroxide. However, when there is a rise in vaginal pH, such as after sex and during menses, bacterial overgrowth could occur. Thus, a composition providing an acidic pH in the vagina after application is advantageous.

The pH regulating agent may be any agent that can adjust the pH to a pH value in a range as mentioned above. Thus, it may be selected from a buffer substance such as an acetate or a lactate. As the active substance is an oligomer of lactic acid, lactate is preferred. Lactate is typically in the form of an alkaline earth metal salt such as sodium or potassium lactate. As seen from the examples, a suitable form of lactate used is an aqueous solution of lactate, where the solvent functions as the liquid basis in the granulation liquid. More specifically, a 50% aqueous solution of sodium lactate is use.

In the first granulation step about 50 to 75% w/w of the total amount of oligomeric lactic acid is dissolved in a 50% aqueous solution of sodium lactate. The amount of the 50% aqueous solution of sodium lactate is in a range of from 50 to 75% w/w of the total amount of 50% aqueous solution of sodium lactate used. Normally, the weight ratio between the 50% aqueous solution of sodium lactate and the oligomeric lactic acid in the first granulation liquid is in a range of from about 0.25 to about 0.7 such as in a range of from about 0.4 to about 0.65 or from about 0.5 to about 0.6.

In the second granulation step about 25 to 50% w/w of the total amount of oligomeric lactic acid is dissolved in a 50% aqueous solution of sodium lactate. The amount of the 50% aqueous solution of sodium lactate is in a range of from 25 to 50% w/w of the total amount of 50% aqueous solution of sodium lactate used. Normally, the weight ratio between the 50% aqueous solution of sodium lactate and the oligomeric lactic acid in the second granulation liquid is in a range of from about 0.25 to about 0.7 such as in a range of from about 0.4 to about 0.65 or from about 0.5 to about 0.6.

It is preferred that the weight ratios between the 50% aqueous solution of sodium lactate and the oligomeric lactic acid in the first and second granulation liquid are the same.

In order to avoid any degradation of oligomeric lactic acid in the granulation liquid it is preferred to use a freshly prepared granulation liquid in a process of the invention.

The wet granulation can be carried out in any suitable apparatus such as a fluid bed dryer or high shear granulation mixer. The inventors have found that good results of the first granulate when using a fluid bed for the wet granulation step.

The wet massing in the first granulation step is performed for a desired time period dependent on the apparatus employed and the amount of product to be granulated. In the event a fluid bed apparatus is employed, the time period depends on the batch scale.

After the first granulation step, the wet granulate is milled. This is typically performed by any suitable method such as sieving through a sieve having the desired mesh. The mesh is typically in a range of from about 1.4 to about 4 mm such as in a range of from about 1.6 to about 3 mm, from about 1.8 to about 2.5 mm such as about 2 mm.

After milling of the wet first granulate, it is subject to drying. The drying may be performed eg in a suitable oven or it may be performed in a fluid bed dryer. The wet massing in the second granulation step is performed for a desired time period desired dependent on the apparatus employed and the amount of product to be granulated.

The drying of the wet first granulate is typically performed until the dried first granulate has a water content of 2.5% or less such as 2.3% or less, 2.2% or less, 2.1% or less or 2.0% or less as measured by loss on drying (LOD).

The drying of the milled wet first granulate is performed at a temperature in a range of from about 25° C. to about 50° C. such as from about 25° C. to about 45° C., or from about 25° C. to about 30° C. If a fluid bed apparatus is employed, the inlet temperature is typically higher such as up to about 50° C. and the product temperature is typically at the most about 25° C. to about 45° C. At the end of the drying the temperature is about 30-35° C.

It is important to ensure that the temperature of the first (and second) granulate is not too high to avoid degradation of the oligomeric lactic acid. Moreover, when the temperature increases the oligomeric lactic acid will tend to melt and then tend to stick to the surfaces of the apparatus used for the drying process.

After drying of the first granulate it is milled to a smaller particle size by sieving the dry second granulate through a sieve having a mesh of from about 1.0 to about 3 mm such as from about 1.2 to about 2.5 mm, from about 1.4 to about 2.0 mm such as about 1.6 mm.

Then the dry first granulate it is subjected to a second wet granulation step. In this step a granulation liquid is used, which has the same composition as the one used in the first granulation step. From about 25% to about 75% w/w such as from about 35% to about 70% w/w, from about 40% to about 70% w/w, from about 50% to about 70% w/w or from about 60% to about 70% w/w of the total weight of the granulation liquid used in the first and second wet granulation steps is used in the first granulation step. The remaining part of the granulation liquid is used in the second granulation step, i.e. from about 25% to about 75% w/w such as from about 30% to about 65% w/w, from about 30% to about 60% w/w, from about 30% to about 50% w/w or from about 30% to about 40% w/w of the total weight of the granulation liquid used in both granulation steps.

The second granulation step may be carried out in any suitable apparatus, typically the same as that used for the first granulation step. A fluid bed or a high shear mixer has proved to be suitable apparatus.

After the second granulation step, the wet granulate is milled. This is typically performed by any suitable method such as sieving through a sieve having the desired mesh. The mesh is typically in a range of from about 1.4 to about 4 mm such as in a range of from about 1.6 to about 3 mm, from about 1.8 to about 2.5 mm such as about 2 mm.

The drying of the wet second granulate is performed at a temperature in a range of from about 25° C. to about 50° C. such as from about 25° C. to about 45° C., or from about 30° C. to about 45° C. If a fluid bed apparatus is employed, the inlet temperature is typically higher such as up to about 50° C. and the product temperature is typically at the most about 25° C. to about 45° C. At the end of the drying the temperature is about 30-35° C.

It is important to ensure that the temperature of the first (and second) granulate is not too high to avoid degradation of the oligomeric lactic acid. Moreover, when the temperature increases the oligomeric lactic acid will tend to melt and then tend to stick to the surfaces of the apparatus used for the drying process.

The drying of the second granulate may be performed in any suitable apparatus such as e.g. an oven or a fluid bed.

The drying of the second granulate is performed until the dried second granulate obtained from step vii) has a water content of 2% or less such as 1.9% or less, 1.8% or less, 1.7% or less, 1.6% or less, 1.5% or less, 1.4% or less, as measured by LOD or a water content of 3% or less such as 2.9% or less, 2.8% or less, 2.7% or less, 2.6% or less, 2.5% or less as measured by Karl Fischer method.

After drying of the second granulate it is milled to a smaller particle size by sieving the dry second granulate through a sieve having a mesh of from about 1.0 to about 3 mm such as from about 1.2 to about 2.5 mm, from about 1.4 to about 2.0 mm such as about 1.6 mm.

The thus obtained particulate material has a particle size distribution corresponding to a mean particle size of from about 200 to about 300 μm.

The particulate material has a poured density in a range of from 0.5 kg/dm³ to about 0.6 kg/dm³ as measured by bulk and tapped density apparatus. The relatively large particle size distribution and the density of the material have shown to be advantageous in the event the particulate material is further processed into a pharmaceutical or cosmetical dosage form. Thus, they contribute to that it is possible to obtain tablet of relatively small size in view of the weight of the tablets as well as it contributes to the mechanical properties of the tablets such as friability, hardness etc. As seen from the Examples herein a tablet can be obtained with a tablet weight of 3.3 g, and dimensions 16.7×25.6 mm. The height depends on tablet hardness but is approx. 10-11 mm.

In order to process the particulate material into a suitable dosage form, additional pharmaceutically acceptable excipients may be added to and admixed with the particulate material. Normally, such excipients include excipients that can impart good flowability or lubrication. Examples include lubricants or glidant.

A lubricant may be added prior to compression, pelletizing or prior to filling of a particulate material into capsules or the like. Lubricants prevent sticking of the tablets to the tablet punches during the compression phase of the tablet manufacturing process.

When lubricants are added to a powder mass, they form a coat around individual particles which remains more or less intact during compression. Lubricants are mostly hydrophobic. The presence of lubricant coating may cause an increase in the disintegration time and a decrease in drug dissolution rate. The choice of a lubricant may depend upon the type of tablet being manufactured, dissolution, flow characteristics and requirements of the formulation in terms of hardness, friability and compatibility. Normally, glidants are regarded as materials that have good flow properties and poor lubrication properties.

Glidants improve the flow of powder into the tableting machines for compaction. They act to minimize the tendency of a granulation to separate or segregate due to excessive vibration. High speed tablet machine requires smooth even flow of material to die cavities (tablet mold). The uniformity of tablet weights directly depends on how uniformly the die cavity is filled. In general, many materials commonly referred to as lubricants possess only a minimal lubricating activity and are better glidants or anti-adherents.

In the present context, the term “lubricant” is used both for lubricants and glidants.

Examples of lubricants suitable for use in the present context include stearates, stearic acid, metallic stearates including magnesium stearate, calcium stearate, talc, waxes, hydrogenated vegetable oils, glycerides, colloidal silica, sodium stearyl fumarate, polyethylene glycols and alkyl sulfates.

The concentration of a lubricant in the final product is typically in a range of from 0% to about 5% w/w such as from 0.1% to about 5% w/w, from about 0.5% to about 4% w/w such as about 1% w/w.

The water content in the final composition (particulate material or dosage form) is typically higher than in the dried second granulate. Thus, it may be in a range of from 2.5% to about 3.5% w/w as determined by Karl Fischer method.

### Use of a Composition of the Invention

A composition of the invention is typically suited to use in the treatment or prevention of a gynecological infection such as those mentioned herein before. Thus, it typically has a form or a shape that is suitable for application to the vagina. Such composition includes vagitoriums, vaginal rods, vaginal discs, tablets etc.

However, OMLA has other therapeutic uses and may be employed in other forms than those mentioned above. Thus, the particulate material may be used as such or it may be admixed with pharmaceutically or cosmetically acceptable excipients with the purpose of obtaining final compositions suitable for use e.g. in dermatological formulations.

### Active Substance

Lactic acid is a hydroxy carboxylic acid with the structure CH₃C(OH)—COOH. Oligomers of lactic acid (OMLA) are formed by esterification of the carboxylic group of a lactic acid with the hydroxyl group of a lactic acid. Thus, oligomers of lactic acid are chains of lactic acids coupled to each other by ester links between the carboxylic acid moiety in one with the secondary alcohol function in another.

OMLA for use in the present invention include all combinations of optical isomers of oligomeric lactic acids (e.g. R and S enantiomers, D- and L-forms as well as racemic, diastereomeric, meso and other mixtures of such isomers). The L-form is preferred and used in the Examples herein.

Oligomers of lactic acid are used for the treatment of gynecological infections such as bacterial, viral or fungal infections and have been used for the treatment of bacterial vaginosis. Moreover, OMLA has been used in the treatment of oral mucosal lesions, it has been used in the field of dermatology in the treatment of wounds, eczemas, atopic dermatitis psoriasis, acne, rosacea, urticaria, pruritus, light dermatosis, hyperhidrosis, alopecia and infections. OMLA has also been used in gastroenterology, where beneficial effects may be seen in acid disorders such as achylia.

The monomer is lactic acid and oligomers of lactic acid are presented by the following formula. If n is 1, then the compound is the dimer of lactic acid.

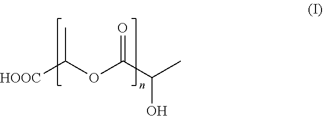

OMLA for use in the present invention typically contains a mixture of different oligomers of lactic acid where n is an integer ranging from 1 to 25 such as e.g. from 2 to 25, from 2 to 20, from 3 to 20, from 2 to 15, from 2 to 10, from 3 to 10. Moreover, in OMLA for use in the present invention lactate may also be present. Thus, OMLA for use in the present invention may contain one or more main oligomers together with a number of oligomers of smaller or larger size. The higher the mean molecular is, the wider is the molecular weight distribution of OMLA.

OMLA for use in the present invention typically has a weight average molecular weight of from about 150 to about 2,000 such as from about 175 to about 2,500, from about 200 to about 2,000, from about 200 to about 1,500, from about 200 to about 1,000. From about 200 to about 750, from about 200 to about 500, from about 225 to about 400, from about 225 to about 350 such as from 230 to 310.

OMLA for use in the present invention typically has a degree of polymerization in a range of from about 2.0 to about 7.0 such as from about 2.5 to about 6.5, from about 2.7 to about 6, from about 2.7 to about 5.5, from about 2.7 to about 5 or from about 3 to about 5.

The term “weight average molecular weight” or “Mw” is intended to describe the molecular weight of on oligomer or polymer. The weight average molecular weight is calculated as Mw=Σᵢ(NᵢM₁²)/Σᵢ(NᵢM₁), wherein Nᵢ, is the number of molecules of molecular weight M₁. If the weight average molecular weight is w and you pick a random monomer, then the polymer it belongs to will have a weight of w on average. The weight average molecular weight can be determined by e.g. mass spectrometry, NMR spectroscopy, light scattering, small angle neutron scattering (SANS), X-ray scattering or sedimentation velocity.

By the term “number average molecular weight” of “Mₙ” is intended to mean a determination of the molecular weight of an oligomer of polymers. The number average molecular weight is the common, mean, average of the molecular weights of the individual oligomers or polymers. It is determined by measuring the molecular weight of n polymer molecules, summing the weights and dividing by n. Mₙ=Σᵢ(NᵢM₁)/Σᵢ(Nᵢ), wherein Nᵢ, is the number of molecules of molecular weight Mᵢ. The number average molecular weight of an oligomer or polymer can be determined by e.g. mass spectrometry, NMR spectroscopy, vapor pressure osmometry, end-group titration or colligative properties.

By the term “polydispersity index” is intended to mean a measure of the distribution of molecular weights in an oligomeric or polymeric sample. It is determined as the ratio of the weight average molecular weight to the number average molecular weight of an oligomer or a polymer.

The term “degree of polymerization” (DP) refers to the number of monomer units there is in the oligomer or polymer. It is calculated as the ratio of molecular weight of an oligomer and molecular weight of the repeat unit and is identical with the polydispersity index defined above.

### BRIEF DESCRIPTION OF THE FIGURES

FIG. 1 is a flow chart of the manufacturing process

FIG. 2 is the dissolution profile of a composition prepared by the process of the invention.

### METHODS

Total lactic acid in a composition prepared by a process of the invention The total content of oligomeric lactic acid in a composition is determined by hydrolysis of the oligomeric lactic acid by 1.0 M sodium hydroxyde, and the solution is then back-titrated pptentiometrically with 1.0 M hydrochloric acid.

The total amount of lactic acid (mmol/g) is calculated as follows:

Calculate the average content with at least 4 decimals of total lactic acid from the duplicate sample and placebo preparations:

$$TA = \frac{( {V}_{NaOH} \times {C}_{NaOH} ) - ( ( {V}_{HCl - EP} + ( 20 - {VP}_{HCl - EP} ) ) \times {C}_{HCl} ) \times 1000}{m}$$

where TA=Total lactic acid mmol/g
V_{NaOH}=Total volume of NaOH added to the sample (mL)
C_{NaOH}=Concentration of NaOH (mol/L)
V_{HCl}=Total volume of HCl used in the titration (mL)
VP_{HCl}=Total volume of HCl used in the Placebo titration (mL)
C_{HCl}=Concentration of HCl (mol/L)
m=Weight of sample (mg)

The total amount of lactic acid (mg lactic acid/unit) is calculated as follows:

Calculate the total amount of lactic acid in mg/unit according to equation below:

$${TA}_{tot} = TA \times {Mw}_{lactic acid} \times m$$

where TAₜₒₜ=Total lactic acid mg/unit
TA=Mean value total lactic acid mmol/g according to 6.1
Mw_{lactic} acid=Molecular weight lactic acid 90.08 (g/mol)
m=Average tablet weight (g) (n=5).

The degree of polymerisation is calculated according to the equation: PD=TA/FA, where FA is the mean value of free lactic acid in mmol/g, and TA is the mean value of total lactic acid in mmol/g after hydrolysis of the oligomeric lactic acid. The free acid of oligomeric lactic acid is determined by potentiometric non-aqueous titration of the acid using 0.1 M potassium methoxide in methanol as titrant and a combined pH glass electrode to determine the end point pH 8.4. Methanol is used as solvent.

### Examples

### Example 1—Process for the Preparation of a Particulate Material According to the Invention

Particulate material and tablets were made using a process according to the invention and based on the following ingredients.

Batch 006-010 were manufactured to evaluate the flowability and compressibility of the granules. Batch 007, 008 and 009 have a similar composition as batch 006 but different grades of lactose and microcrystal line cellulose were used. In batch 007 lactose grade 450M was used, which has a smaller particle size (D50:20 μm) than 200M (D50:40 μm). In batch 008, microcrystalline cellulose (MOO) grade PH105 was used with a smaller particle size (about 20 μm) compared to PH101 (about 50 μm) as used in batch 006. In batch 009 both grades of lactose and MOO with smaller particle size were used together.

Formulation A (batch 006)

| | |
|---|---|
| % w/w | Amount/tablet (mg) |

Intra-granular components for preparation of a particulate material

| | | |
|---|---|---|
| Oligomeric lactic acid | 17.3 | 693. |
| (Purasorb OL)* | | |
| Hypromellose (Pharmacoat | 40.7 | 1628 |
| 615) | | |
| Lactose monohydrate | 19.6 | 784 |
| (Pharmatose 200M) | | |
| Microcrystalline cellulose | 15.3 | 611 |
| (Avicel PH101) | | |
| Sodium lactate solution 50% | 6.1 | 244 |

Extra-granular components for tablet preparation

| | | |
|---|---|---|
| Magnesium stearate | 1.0 | 41 |
| Total | 100.0 | 4,000 |

396 mg oligomeric lactic acid corresponds to 809 mg lactic acid

Formulation B (Batch 010)

| | |
|---|---|
| % w/w | Amount/tablet (mg) |

Intra-granular components:

| | | |
|---|---|---|
| Oligomeric lactic acid | 17.3 | 693. |
| (Purasorb OL)* | | |
| Hypromellose (Pharmacoat | 40.7 | 1628 |
| 615) | | |
| Lactose monohydrate | 34.9 | 1394 |
| (Pharmatose 200M) | | |
| Sodium lactate solution 50% | 6.1 | 244 |

Extra-granular components for tablet preparation

| | | |
|---|---|---|
| Magnesium stearate | 1.0 | 41 |
| Total | 100.0 | 4,000 |

396 mg oligomeric lactic acid corresponds to 809 mg lactic acid

The flowability of formulations A and B in particulate form was evaluated by means of bulk and tapped density using a jolting volumeter (STAV 2003) and calculating Hausner ratio. A hausner ratio between 1.12 and 1.18 is an indication of excellent flowability. The particle size distribution (PSD) was measured by means of laser diffraction using Malvern, Mastersizer 3000.

The results obtained were:

| | Poured density | Hausner | D50 |
|---|---|---|---|
| Batch No. | (kg/dm³) | ratio | (μm) |
| 006 | 0.56 | 1.25 | 112 |
| 007 | 0.55 | 1.23 | 125 |
| 008 | 0.60 | 1.21 | 132 |
| 009 | 0.60 | 1.19 | 140 |
| 010 | 0.64 | 1.14 | 175 |

The batches 006, 007, 008, 009 and 010 were made using only one granulation step. In order to decrease the tablet size the following compositions were made:

Formulation C (batch 015)

| | |
|---|---|
| % w/w | Amount/tablet (mg) |

Intra-granular components:

| | | |
|---|---|---|
| Oligomeric lactic acid | 16.8 | 620 |
| (Purasorb OL)* | | |
| Hypromellose (Pharmacoat | 41.8 | 1545 |
| 615) | | |
| Lactose monohydrate 314 | 34.7 | 1284 |
| rennet free | | |
| Sodium lactate solution 50% | 5.6 | 209 |

Extra-granular components for tablet preparation

| | | |
|---|---|---|
| Magnesium stearate | 1.1 | 42 |
| Total | 100.0 | 3,700 |

620 mg oligomeric lactic acid corresponds to 724 mg lactic acid

Formulation D (batch 022)

| | |
|---|---|
| % w/w | Amount/tablet (mg) |

Intra-granular components:

| | | |
|---|---|---|
| Oligomeric lactic acid | 17.1 | 581 |
| (Purasorb OL)* | | |
| Hypromellose (Pharmacoat | 42.6 | 1447 |
| 615) | | |
| Lactose monohydrate 314 | 33.5 | 1138 |
| rennet free | | |
| Sodium lactate solution 50% | 5.6 | 191 |

Extra-granular components for tablet preparation

| | | |
|---|---|---|
| Magnesium stearate | 1.3 | 43 |
| Total | 100.0 | 3,400 |

581 mg oligomeric lactic acid corresponds to 679 mg lactic acid

Formulation E (batch 024)

| | |
|---|---|
| % w/w | Amount/tablet (mg) |

Intra-granular components:

| | | |
|---|---|---|
| Oligomeric lactic acid | 19.5 | 642 |
| (Purasorb OL)* | | |
| Hypromellose (Pharmacoat | 25.0 | 825 |
| 615) | | |
| Lactose monohydrate 314 | 49.1 | 1620 |
| rennet free | | |
| Sodium lactate solution 50% | 5.5 | 180 |

Extra-granular components for tablet preparation

| | | |
|---|---|---|
| Magnesium stearate | 1.0 | 33 |
| Total | 100.0 | 3,300 |

642 mg oligomeric lactic acid corresponds to 750 mg lactic acid

The batches 015, 022 and 024 were made employing the two step granulation procedure. The flowability of formulation Ein particulate form was evaluated by means of bulk and tapped density using a jolting volumeter (STAV 2003) and calculating hausner ratio. A hausner ratio between 1.12 and 1.18 is an indication of excellent flowability. The particle size distribution (PSD) was measured by means of laser diffraction using Malvern, Mastersizer 3000.

The results obtained were:

| | Poured density | | D50 |
|---|---|---|---|
| Batch No. | (kg/dm³) | Hausner ratio | (μm) |
| 024 | 0.63 | 1.17 | 238 |

The manufacturing process was as follows:

To obtain the granulation liquid, the oligomeric lactic acid was heated in a water bath at approx. 40-50° C. under stirring to decrease its viscosity. The sodium lactate solution was added. The granulation liquid was kept in the water bath but was divided into a first granulation and a second granulation liquid.

The other intra-granular ingredients were filled into a fluid bed apparatus and the first part of the granulation liquid was added gradually to the powder mixture. When a homogeneous wetted powder was obtained it was sieved through a 2 mm mesh and dried in the fluid bed at 50° C., and the dry first granulate was sieved through a 1.6 mm mesh. Then the second granulation liquid was added gradually to the mixture and the wet granules obtained were wet milled (as it was done for the first granulate) and then dried in the fluid bed followed by sieving through a 1.6 mm mesh at 3000 rpm to remove lumps. The moisture content was measured by means of loss on drying until the water content was 2.5% or less.

The particulate material obtained was mixed with magnesium stearate and compressed into tablets.

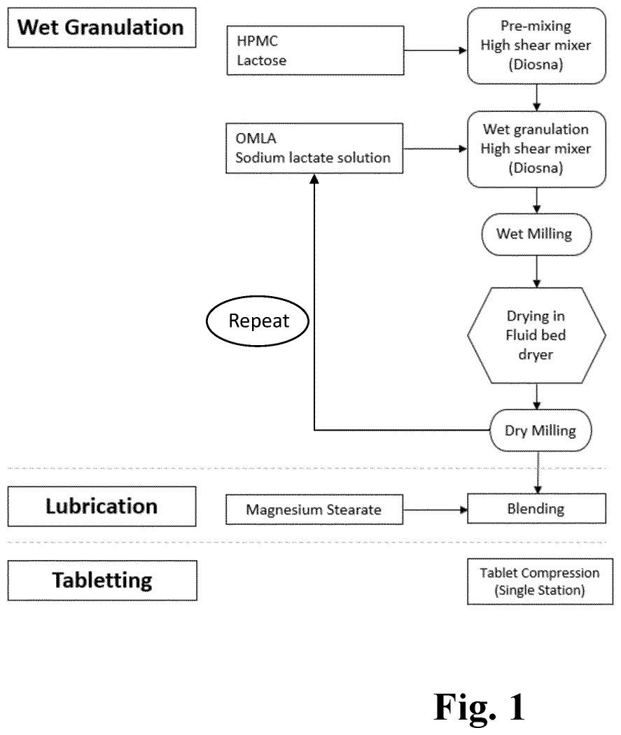

A flow chart of the process is seen in FIG. 1.

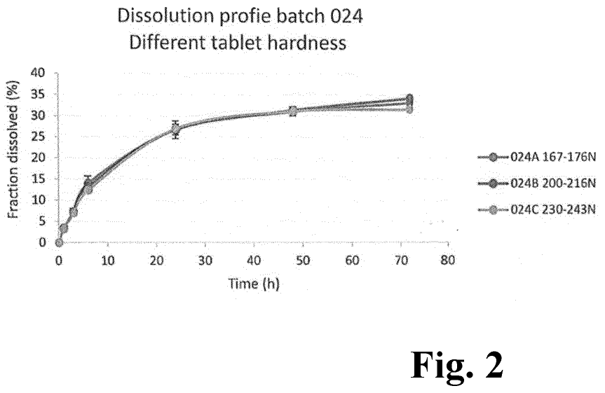

The release of lactic acid from batch 024 was investigated. The in-vitro dissolution is performed in 900 mL 0.9% NaCl at 37° C. using paddles set at 100 rpm. At fixed time intervals, 0.5 M NaOH is added to raise the pH to 5.0-5.5. The dissolution is followed for from 2 to 96 h and is evaluated as total amount of base added divided by the theoretical amount needed to hydrolyse one vaginal tablet. The results appear from FIG. 2.

### Example 2—Stability Testing

A formulation prepared according to the invention was subject to stability testing.

Formulation F (batch D2057-027):

| | |
|---|---|
| % w/w | Amount/tablet (mg) |

Intra-granular components:

| | | |
|---|---|---|
| Oligomeric lactic acid | 19.5 | 642 |
| (Purasorb OL)* | | |
| Hypromellose (Pharmacoat | 25.0 | 825 |
| 615) | | |
| Lactose monohydrate 314 | 49.1 | 1620 |
| rennet free | | |
| Sodium lactate solution 50% | 5.5 | 180 |

Extra-granular components for tablet preparation

| | | |
|---|---|---|
| Magnesium stearate | 1.0 | 33 |
| Total | 100.0 | 3300 |

642 mg oligomeric lactic acid corresponds to 750 mg lactic acid

A batch size of 1.4 kg was used.

The two-steps granulation process was applied. Lactose and Hypromellose was pre-blended with tip speed 4.1 m/s for approx. 3 min. In the first granulation round 290 g of the granulation liquid containing both oligomeric lactic acid and sodium lactate solution was added to the powder. Wet massing was performed for 1 m. The wet granulate was then wet milled manually through a 2 mm mesh before drying. Thue fluid bed dryer used was an Aeromatic Fielder AG (model Strea1) with container volume 16.5 L. The drying time and process parameters for two separate rounds of the first granulation step is given in the tables below. The moisture content was only measured with LOD in the first granulation round.

Fluid bed drying process parameters, granulation 1, round 1

| Time (min) | Product temp. ° C. | Airflow (cfm) | Inlet temp. ° C. |
|---|---|---|---|
| Initially | 34 | 2 | 63 |
| 5 | 42 | 2 | 57 |
| 8 | 33 | 2 | 33 |
| 10 | 32 | 2 | 33 |

Fluid bed drying process parameters, granulation 1, round 2

| Time (min) | Product temp. ° C. | Airflow (cfm) | Inlet temp. ° C. |
|---|---|---|---|
| Initially | 21 | 2 | 28 |
| 3 | 24 | 2 | 29 |
| 5 | 24 | 2 | 27 |
| 10 | 29 | 2 | 34 |
| 15 | 30 | 2 | 31 |

LOD measurements from granulation 1, round 1 and 2

| Round | Drying time (min) | Water content (%) |
|---|---|---|
| 1 | 5 | 1.91 |
| 1 | 10 | 1.62 |
| 2 | 5 | 2.48 |
| 2 | 15 | 2.48 |

After drying, the granules were milled through a 1.4 mm screen using a conical mill (Uni-Mill M05-U, Hanningfield, UK). The granules were then added to the fluid bed for the second granulation round. In the second granulation round the remaining granulation liquid was added up to 425 g. Wet massing was only performed fir 15 sec. The wet granules were subsequently wet milled manually through a 2 mm mesh, dried and milled through a 1.4 mm screen.

The granules from the second granulation round had a higher moisture content—hence a higher density. Both LOD and Karl Fischer were used to measure the water content. The product temperature was kept below 50° C. to not affect the stability of the oligomeric lactic acid.

Fluid bed dryer process parameters, granulation 2, round 1

| Time (min) | Product temp. ° C. | Airflow (cfm) | Inlet temp. ° C. |
|---|---|---|---|
| Initially | 21 | 2 | 35 |
| 2 | 33 | 7 | 46 |
| 3 | 33 | 6 | 41 |
| 5 | 33 | 6 | 38 |
| 8 | 33 | 5 | 35 |
| 10 | 33 | 5 | 34 |
| 15 | 33 | 5 | 34 |
| 25 | 32 | 5 | 33 |
| 45 | 38 | 4 | 40 |
| 55 | 33 | 4 | 33 |
| 65 | 32 | 4 | 33 |
| 80 | 32 | 4 | 33 |

Water content measure with LOD and Karl
Fischer, granulation 2, round 1

| Round | Drying time (min) | LOD (%) | Karl Fischer (%) |
|---|---|---|---|
| 1 | Initially | — | 4.18 |
| 1 | 5 | 1.89 | 2.81 |
| 1 | 15 | 1.76 | 2.40 |
| 1 | 35 | 1.32 | 2.74 |
| 1 | 55 | 1.17 | 2.79 |
| 1 | 80 | — | 2.59 |

The average water content from 5-80 mm was 2.6%. pH was stable at pH 3.3 during the drying process confirming the stability of oligomeric lactic acid. Some of the wet granulate had been stored in a plastic bag for 48 h and had dried a bit, it could be divided into 2 parts. The drying process parameters and LOD for round 2 and 3 are presented in the table below. A higher inlet temperature up to 50° C. was used without the granulate sticking to the bottom.

Fluid bed dryer process parameters, granulation 2, round 2 and 3

| | | Product temp. | Airflow | Inlet temp. |
|---|---|---|---|---|
| Round | Time (min) | ° C. | (cfm) | ° C. |
| 2 | Initially | 25 | 5 | 44 |
| 2 | 17 | 33 | 5 | 39 |
| 2 | 30 | 46 | 5 | 53 |
| 3 | Initially | 30 | 5 | 47 |
| 3 | 15 | 48 | 5 | 51 |
| 3 | 30 | 46 | 5 | 47 |

Water content measured with LOD. Granulation 2, round 2 and 3

| Round | Drying time (min) | LOD (%) |
|---|---|---|
| 2 and3 | Initially | 2.37 |
| 2 | 17 | 1.27 |
| 2 | 30 | 0.96 |
| 3 | 15 | 1.02 |
| 3 | 30 | 0.76 |

Since lactose and Hypromellose are the major components in the formulation, the water content of both raw materials was measured with Karl Fischer before and after drying in a drying cabinet at 60° C. for 2 h. The results show that the water content in lactose could not be decreased at the drying temperature used for the formulation. In theory, this means that the granulate for the formulation cannot obtain a water content below 2.4% since it contains 49% lactose. The drying process was hence stopped with a water content of 2.6%.

| Round | Water content before (%) | Water content after (%) |
|---|---|---|
| Hypromellose | 1.96 | 0.52 |
| Lactose | 4.92 | 4.96 |

Finally, the particulate material obtained was mixed with magnesium stearate (lubricant). The target tablet weight was obtained at 3.3 g with a tablet hardness between 180-210 N as desired. Each tablet was packed in an aluminium bag with double sealing for stability testing.

A friability test was also performed using a Pharma test Fribilator PTF E. The tablet weight before was 33.006 g ad after 32.768 g hence the friability was calculated to 0.72% (limit 1%).

The results of the stability tests are shown in the following.

Batch D 2057-027
Storage at 25° C./60% RH

| | | | | | | | | 12 | 18 |
|---|---|---|---|---|---|---|---|---|---|
| Analysis | Limits | Initial | 1 month | 2 months | 3 months | 6 months | 9 months | months | months |
| Visual | White to | Conforms | Conforms | Conforms | Conforms | Conforms | Conforms | Conforms | Conforms |
| inspection | off- | | | | | | | | |
| | white, | | | | | | | | |
| | oblong | | | | | | | | |
| | tablet. | | | | | | | | |
| | No | | | | | | | | |
| | foreign | | | | | | | | |
| | particles | | | | | | | | |
| | or | | | | | | | | |
| | debris | | | | | | | | |
| Mean Tablet | | 3.31 | 3.31 | 3.32 | 3.31 | 3.31 | 3.31 | 3-32 | 3-25 |
| weight (5 | | | | | | | | | |
| tablets) g | | | | | | | | | |
| Total lactic | | 867 | 850 | 868 | 858 | 854 | 871 | 878 | 830 |
| acid, mg/unit | | | | | | | | | |
| Degree of | 3.0-5.0 | 3.6 | 3.5 | 3.5 | 3.4 | 3.3 | 3.3 | 3-3 | 3.1 |
| polymerization | | | | | | | | | |
| Free lactic | | 241 | 241 | 249 | 250 | 257 | 263 | 268 | 268 |
| acid mg/unit | | | | | | | | | |
| pH | 3.0-4.0 | 3.4 | 3.4 | 3.4 | 3.4 | 3.4 | 3.3 | 3.3 | 3.3 |
| Water | | 2.8 | 3.0 | 3.1 | 2.9 | 2.9 | 2.8 | 2-9 | 2.9 |
| content, % | | | | | | | | | |
| Dissolution | | | | | | | | | |
| profile, % | | | | | | | | | |
| lactic acid | | | | | | | | | |
| dissolved at: | | | | | | | | | |
| 6 hours | | 11 | 12 | 8 | 12 | 12 | 9 | 9 | 7 |
| (range) | | (10-12) | (11-12) | (6-9) | (10-13) | (10-12) | (9-10) | (8-10) | (5-8) |
| 24 hours | | 26 | 26 | 25 | 27 | 27 | 26 | 28 | 23 |
| (range) | | (24-28) | (25-27) | (24-25) | (25-28) | (26-28) | (25-27) | (27-29) | (22-24) |
| 48 hours | | 31 | 29 | 30 | 29 | 30 | 31 | 32 | 26 |
| (range) | | (30-32) | (29-30) | (29-31) | (28-31) | (29-30) | (29-32) | (31-33) | (24-27) |
| 72 hours | | 33 | 32 | 30 | 31 | 32 | 33 | 34 | 26 |
| (range) | | (32-33) | (31-32) | (29-31) | (30-33) | (31-33) | (31-33) | (32-35) | (24-29) |

Batch D 2057-027
Storage at 40° C./75% RH

| Analysis | Limits | Initial | 1 month | 2 months | 3 months | 6 months |
|---|---|---|---|---|---|---|
| Visual | White to | Conforms | Conforms | Conforms | Conforms | Conforms |
| inspection | off- | | | | | |
| | white, | | | | | |
| | oblong | | | | | |
| | tablet. | | | | | |
| | No | | | | | |
| | foreign | | | | | |
| | particles | | | | | |
| | or | | | | | |
| | debris | | | | | |
| Mean Tablet | | 3.31 | 3.31 | 3.32 | 3.30 | 3.31 |
| weight (5 | | | | | | |
| tablets) g | | | | | | |
| Total lactic | | 867 | 822 | 869 | 862 | 867 |
| acid, mg/unit | | | | | | |
| Degree of | 3.0-5.0 | 3.6 | 3.0 | 3.2 | 3.0 | 3.0 |
| polymerization | | | | | | |
| Free lactic | | 241 | 277 | 278 | 289 | 291 |
| acid mg/unit | | | | | | |
| pH | 3.0-4.0 | 3.4 | 3.3 | 3.2 | 3.2 | 3.2 |
| Water content, | | 2.8 | 2.8 | 2.9 | 2.8 | 2.8 |
| % | | | | | | |
| Dissolution | | | | | | |
| profile, % | | | | | | |
| lactic acid | | | | | | |
| dissolved at: | | | | | | |
| 6 hours | | 11 | 14 | 10 | 14 | 14 |
| (range) | | (10-12) | (12-15) | (7-14) | (13-15) | (13-15) |
| 24 hours | | 26 | 29 | 30 | 32 | 31 |
| (range) | | (24-28) | (28-29) | (29-33) | (32-33) | (30-32) |
| 48 hours | | 31 | 31 | 34 | 35 | 35 |
| (range) | | (30-32) | (31-33) | (33-35) | (34-36) | (32-36) |
| 72 hours | | 33 | 34 | 34 | 36 | 36 |
| (range) | | (32-33) | (33-36) | (33-35) | (35-38) | (34-38) |

Batch D 2057-027
Storage at 50° C./75% RH

| Analysis | Limits | Initial | 1 month |
|---|---|---|---|
| Visual inspection | White to | Conforms | Conforms |
| | off-white, | | |
| | oblong | | |
| | tablet. No | | |
| | foreign | | |
| | particles | | |
| | or debris | | |
| Mean Tablet | | 3.31 | 3.32 |
| weight | | | |
| (5 tablets) g | | | |
| Total lactic acid, | | 867 | 838 |
| mg/unit | | | |
| Degree of | 3.0-5.0 | 3.6 | 3.0 |
| polymerization | | | |
| Free lactic acid | | 241 | 287 |
| mg/unit | | | |
| pH | 3.0-4.0 | 3.4 | 3.2 |
| Water content, % | | 2.8 | 2.8 |
| Dissolution profile, | | | |
| % lactic acid | | | |
| dissolved at: | | | |
| 6 hours | | 11 (10-12) | 16 (12-22) |
| (range) | | | |
| 24 hours | | 26 (24-28) | 31 (29-35) |
| (range) | | | |
| 48 hours | | 31 (30-32) | 34 (33-36) |
| (range) | | | |
| 72 hours | | 33 (32-33) | 36 (35-37) |
| (range) | | | |

1. A process for preparing a composition comprising oligomers of lactic acid as an active substance, the process comprising steps of:
i) premixing pharmaceutically acceptable excipients comprising one or more selected from the group consisting of a binder and a filler to obtain a premix;
ii) a first wet granulation step comprising adding a first aqueous solution comprising oligomers of lactic acid to the premix to obtain a wet first granulate;
iii) milling the wet first granulate to obtain a milled wet first granulate;
iv) drying the milled wet first granulate and dry milling to obtain a dried first granulate;
v) a second wet granulation step comprising adding a second aqueous solution comprising oligomers of lactic acid to the dried first granulate to obtain a wet second granulate;
vi) milling the wet second granulate to obtain a milled wet second granulate;
vii) drying the milled wet second granulate to obtain a dried second granulate; and
viii) milling the dried second granulate to obtain a particulate material comprising the oligomers of lactic acid.

2. The process according to claim 1, further comprising a step of processing the particulate material into a dosage form.

3. The process according to claim 2, wherein the dosage form is a tablet.

4. The process according to claim 1, wherein the dried first granulate has a water content of 2.5% as measured by loss on drying (LOD).

5. The process according to claim 1, wherein the dried second granulate has a water content of 2% or less such as measured by Karl Fischer method.

6. The process according to claim 1, wherein the drying of the milled wet first granulate is performed at a temperature of about 25° C. to about 50° C.

7. The process according to claim 1, wherein the drying of the wet second granulate is performed at a temperature in of about 25° C. to about 50° C.

8. The process according to claim 1, wherein a water content of the composition is about 3% or less as measured by Karl Fischer method.

9. The process according to claim 1, wherein the first aqueous solution and the second aqueous solution further comprise a.

10. The process according to claim 1, wherein the first aqueous solution and the second aqueous solution have a same composition.

11. The process according to claim 1, wherein about 25 to about 75% w/w of a total weight of a granulation liquid used in the first and second wet granulation steps is used in the first wet granulation step.

12. The process according to claim 1, wherein the binder is selected from celluloses.

13. The process according to claim 1, wherein the filler is selected from lactoses.

14. The process according to claim 1, wherein wet granulation in the first wet granulation step and/or the second wet granulation step is performed with a granulation liquid, and wherein a weight ratio between 50% aqueous solution of sodium lactate and oligomeric lactic acid in a first and/or second granulation liquid is about 0.25 to about 0.7.

15. The process according to claim 1, wherein the binder is hydroxypropyl methylcellulose.

16. The process according to claim 1, wherein the filler is lactose rennet free.
